(19) United States
(12) Patent Application Publication
(10) Pub. No.: US 2019/0008878 A1
(43) Pub. Date: Jan. 10, 2019

(54) COMPOSITIONS AND METHOD FOR REDUCING CARDIOTOXICITY

(71) Applicant: SignPath Pharma Inc., Sandy, UT (US)

(72) Inventor: Annie Bouchard, Stoke (CA)

(21) Appl. No.: 16/028,112

(22) Filed: Jul. 5, 2018

Related U.S. Application Data
(60) Provisional application No. 62/529,980, filed on Jul. 7, 2017.

(51) Int. Cl. A61K 31/665; A61K 31/683; A61P 9/04; A61P 9/10; A61P 9/12

(52) U.S. Cl. CPC A61K 31/665; A61K 31/683; A61P 9/04; A61K 45/06; A61P 9/12; A61K 2300/00; A61P 9/10

(57) ABSTRACT

The present invention includes composition and methods for inhibiting or decreasing impaired systolic ejection fraction associated with cardiotoxic therapeutic treatment in a subject receiving a cardiotoxic chemotherapeutic agent causing impaired systolic ejection fraction comprising: identifying a subject in need of cardioprotection from the therapeutic treatment; and delivering an effective amount of a phospholipid or derivatives thereof that is cardioprotective to the heart of the subject thereby inhibiting or decreasing impaired systolic ejection fraction associated with administration of the cardiotoxic chemotherapeutic treatment to the subject.

### CROSS-REFERENCE TO RELATED APPLICATIONS

This application claims priority to U.S. Provisional Application Ser. No. 62/529,980 filed Jul. 7, 2017, the entire contents of which are incorporated herein by reference.

### STATEMENT OF FEDERALLY FUNDED RESEARCH

None.

### TECHNICAL FIELD OF THE INVENTION

The present invention relates in general to the field of cardiotoxicity, and more particularly, to composition and methods that include phospholipids and phospholipid derivatives, including phosphatidylglycerol and compounds containing phosphatidylglycerol to reduce or eliminate cardiotoxicity, including, improving survival from treatment with cardiotoxic pharmaceutical agents.

### BACKGROUND OF THE INVENTION

Without limiting the scope of the invention, its background is described in connection with cardiotoxic pharmaceutical agents.

There are numerous pharmaceutical agents designed for the treatment of various diseases which are commonly prescribed, despite being known or suspecting of having adverse effects on the patient's heart. In addition to cardiac arrhythmias, including QT prolongation, supraventricular tachycardias (SVT), and atrial fibrillation (AF), a number of other cardiac toxicities can occur, including cardiomyopathy, congestive heart failure, and left ventricular hypertrophy (LVH.)

The cardiotoxicity of those pharmaceutical agents can lead to significant complications that can affect patients being treated for various malignancies. The severity of such toxicity depends on many factors such as the immediate and cumulative dose, the method of administration, the presence of any underlying cardiac condition, and various congenital or acquired cardiac risk factors unique to a particular patient. Moreover, toxicity can be affected by current or previous treatment with other pharmaceutical agents. Cardiotoxic effects can occur immediately during administration of the drug, or they may not manifest themselves until months or years after the patient has been treated.

High-dose chemotherapy remains the therapy of choice for aggressive malignancies. Countless clinical studies have demonstrated that it can significantly prolong patient survival; however, its use and effectiveness are limited by significant side effects, in particular cardiotoxicity. In mid-to-late phase cardiac toxicity, heart failure can appear many years after chemotherapy has ended. Treatment with chemotherapeutic agents is known to result in pericardial and endomyocardial fibrosis, heart failure, myocarditis, or pericarditis. Chemotherapy has also been associated with hemorrhagic myocardial necrosis and cardiomyopathy.

In addition, antineoplastic monoclonal antibodies are also linked to cardiotoxicity.

Infusion-related cardiotoxic effects, such as left ventricular dysfunction, congestive heart failure, and other cardiac dysfunction can occur. The risk of such complications increases if the patient has preexisting cardiac disease, older age, prior cardiotoxic therapy, or radiation to the chest.

Tyrosine Kinase inhibitors (TKIs) have well known cardiotoxic effects. The antracyclins, trastuzumab, imatinib mesylate, dasatinib, nilotinib, sunitinib, sorafenib and lapatinib have all been associated with a range of mechanical and electrical dysfunctions. Among the toxic effects associated with TKIs are QT prolongation, sudden cardiac death (both considered rhythmic dysfunctions), as well as contractility issues such as reduction in left ventricular ejection fraction (LVEF), congestive heart failure (CHF), acute coronary disease, hypertension, and myocardial infarction (MI). Given the therapeutic potential of drugs such as the tyrosine kinase inhibitors, various strategies have been used to attempt to mitigate the cardiotoxicity of cancer treatment. The primary method for preventing cardiac toxicity is to limit the dose of cardiotoxic drugs. There is also some evidence that the method of drug administration may affect the risk of cardiac toxicity. Rapid administration of cardio toxic agents results in high blood levels, which may cause more heart damage than the same amount of drug given over a longer period of time. Giving smaller doses of drug more frequently can also decrease the toxicity compared to large doses of drugs at longer intervals.

The risk of cardiac toxicity from certain chemotherapy agents has been reduced by encapsulating these drugs in a liposome. For example, studies indicate that cardiotoxicity is considerably lower with liposomal doxorubicin formulations than with conventional doxorubicin.

Dexrazoxane is an aminopolycarboxylic acid that has been shown to prevent or reduce the severity of heart damage caused by doxorubicin. Dexrazoxane is thought to protect the heart muscle by blocking the formation of oxygen free radicals. One of the ways that radiation and chemotherapy drugs damage cells is by forming free radicals. Free radicals are unstable molecules that are formed during many normal cellular processes that involve oxygen, such as burning fuel for energy. They are also formed from exposure to elements in the environment, like tobacco smoke, radiation and chemotherapy drugs.

Thus, a need remains for compositions and method for reducing the cardiotoxic effects of drugs or treatments, such as chemotherapeutic and/or post-chemotherapeutic cardiotoxicity.

### SUMMARY OF THE INVENTION

In one embodiment, the present invention includes a method for inhibiting or decreasing impaired systolic ejection fraction associated with cardiotoxic therapeutic treatment in a subject receiving a cardiotoxic chemotherapeutic agent causing impaired systolic ejection fraction comprising: identifying a subject in need of cardioprotection from the cardiotoxic therapeutic agent or treatment; and delivering an effective amount of one or more phospholipids that is cardioprotective to the heart of the subject thereby inhibiting or decreasing impaired systolic ejection fraction associated with administration of the cardiotoxic therapeutic treatment to the subject. In one aspect, the cardiotoxic therapeutic treatment is chemotherapy. In another aspect, the one or more phospholipids prevent post-therapeutic cardiotoxicity. In another aspect, the one or more phospholipids is provided at least one of: before, during, or after the cardiotoxic therapeutic treatment. In another aspect, the one or more phospholipids is a phosphatidylglycerol that is delivered in combination with an existing patient care paradigm for cardiovascular disease. In another aspect, the existing patient care paradigm is selected from treatment with at least one of: antracyclins, doxorubicin, dasatinib, imatinib mesylate, lapatinib, nilotinib, sorafenib, sunitinib, or trastuzumab. In another aspect, the one or more phospholipids is a phosphatidylglycerol that is delivered at the same time as administration of the cardiotoxic therapeutic treatment. In another aspect, the one or more phospholipids is a phosphatidylglycerol that inhibits at least one of pericardial fibrosis, endomyocardial fibrosis, heart failure, hemorrhagic myocardial necrosis, cardiomyopathy, myocarditis, reduction in left ventricular ejection fraction (LVEF), congestive heart failure (CHF), acute coronary disease, hypertension, myocardial infarction, or pericarditis. In another aspect, the cardiotoxic therapeutic treatment is chemotherapy with sunitinib and doxorubicin. In another aspect, the one or more phospholipids is a phosphatidylglycerol containing compounds comprises 1,2-Dimyristoyl-sn-glycero-3-phosphorylglycerol (DMPG). In another aspect, the cardiotoxic therapeutic treatment is with a tyrosine kinase inhibitor. In another aspect, the tyrosine kinase inhibitor is selected from the group consisting of canertinib (CI 1033), erlotinib, gefitinib, imatinib mesylate, leflunomide (SU101), lapatinib, semaxinib (SU5416), sorafenib (BAY 43-9006), sunitinib, vatalanib (PTK787/ZK222584), vandetanib; ZD6474), and combinations thereof In another aspect, the cardiotoxic therapeutic treatment is a radiotherapeutic agent is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof. In another aspect, the cardiotoxic therapeutic treatment is monoclonal antibody is selected from the group consisting of, alemtuzumab, bevacizumab, cetuximab, gemtuzumab, panitumumab, rituximab, tositumomab, trastuzumab and combinations thereof. In another aspect, the cardiotoxicity reduced or mitigated is at least one of: a decrease in the left ventricular ejection fraction, ejection velocity, chronic heart failure, or congestive heart failure. In another aspect, the phospholipid does not encapsulate the cardiotoxic therapeutic agent. In another aspect, the compound has the following structural formula:

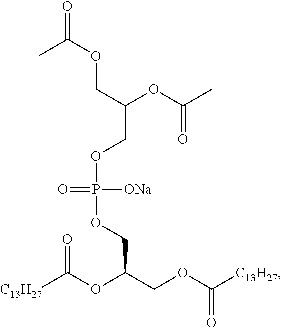

salts or solvates thereof;

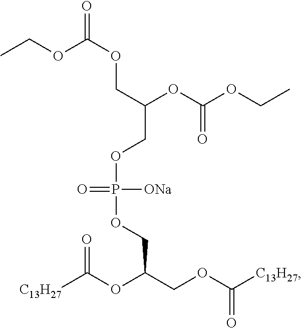

salts or solvates thereof;

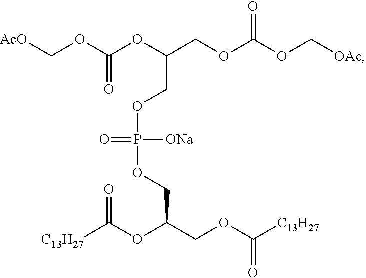

salts or solvates thereof;

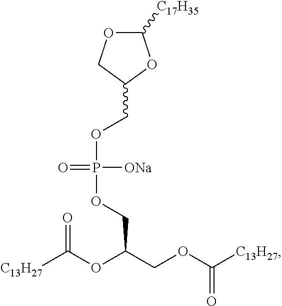

salts or solvates thereof;

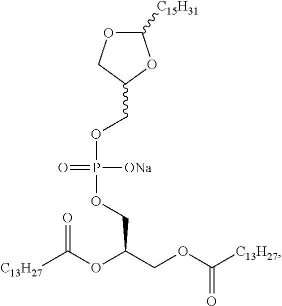

salts or solvates thereof; or

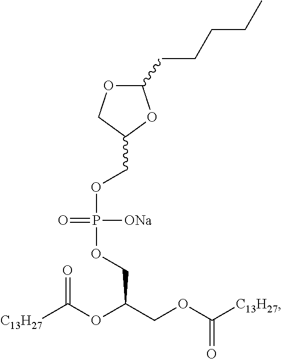

salts or solvates thereof In one aspect, the salt of the conjugate is selected from the group consisting of an acetate, L-aspartate, besylate, bicarbonate, carbonate, D-camsylate, L-camsylate, citrate, edisylate, formate, fumarate, gluconate, hydrobromide/bromide, hydrochloride/chloride, D-lactate, L-lactate, D,L-lactate, D,L-malate, L-malate, mesylate, pamoate, phosphate, succinate, sulfate, bisulfate, D-tartrate, L-tartrate, D,L-tartrate, meso-tartrate, benzoate, gluceptate, D-glucuronate, hybenzate, isethionate, malonate, methylsufate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methane sufonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate, sodium, potassium, calcium, magnesium, zinc, aluminum, lithium, cholinate, lysinium, ammonium, tromethamine, or a mixture thereof. In another aspect, the compound is present in an amount per unit dose of between about 1 mg and about 200 mg per unit dose. In another aspect, the compound is formulated for oral, sublingual, transdermal, suppository, intrathecal, enteral, parenteral, intravenous, intraperitoneal, cutaneous, subcutaneous, topical, pulmonary, rectal, vaginal, or intramuscular administration. In another aspect, the composition formulated for oral administration is a tablet, capsule, caplet, pill, powder, troche, lozenge, slurry, liquid solution, suspension, emulsion, elixir or oral thin film (OTF). In another aspect, the composition in a solid form, a solution, a suspension, or a soft gel form. In another aspect, the solid form further comprises one or more excipients, binders, anti-adherents, coatings, disintegrants, fillers, flavors, dyes, colors, glidants, lubricants, preservatives, sorbents, sweeteners, derivatives thereof, or combinations thereof. In another aspect, the binder is selected from the group consisting of hydroxypropylmethylcellulose, ethyl cellulose, povidone, acrylic and methacrylic acid co-polymers, pharmaceutical glaze, gums, and milk derivatives. In another aspect, the composition further comprises one or more agents that induce a cardiopathy as a side effect, wherein the compound reduces or eliminates the cardiopathy. In another aspect, the one or more agents that induce a cardiopathy as a side effect are selected from at least one of: Albuterol, Alfuzosin, Amantadine, Amiodarone, Amisulpride, Amitriptyline, Amoxapine, Amphetamine, Anagrelide, Apomorphine, Arformoterol, Aripiprazole, Arsenic trioxide, Astemizole, Atazanavir, Atomoxetine, Azithromycin, Bedaquiline, Bepridil, Bortezomib, Bosutinib, Chloral hydrate, Chloroquine, Chlorpromazine, Ciprofloxacin, Cisapride, Citalopram, Clarithromycin, Clomipramine, Clozapine, Cocaine, Curcumin, Crizotinib, Dabrafenib, Dasatinib, Desipramine, Dexmedetomidine, Dexmethylphenidate, Dextroamphetamine, Amphetamine, Dihydroartemisinin and Piperaquine, Diphenhydramine, Disopyramide, Dobutamine, Dofetilide, Dolasetron, Domperidone, Dopamine, Doxepin, Dronedarone, Droperidol, Ephedrine, Epinephrine, Adrenaline, Eribulin, Erythromycin, Escitalopram, Famotidine, Felbamate, Fenfluramine, Fingolimod, Flecainide, Fluconazole, Fluoxetine, Formoterol, Foscarnet, Fosphenytoin, Furosemide, Frusemide, Galantamine, Gatifloxacin, Gemifloxacin, Granisetron, Halofantrine, Haloperidol, Hydrochlorothiazide, Ibutilide, Iloperidone, Imipramine, Melipramine, Indapamide, Isoproterenol, Isradipine, Itraconazole, Ivabradine, Ketoconazole, Lapatinib, Levalbuterol, Levofloxacin, Levomethadyl, Lisdexamfetamine, Lithium, Mesoridazine, Metaproterenol, Methadone, Methamphetamine, Methylphenidate, Midodrine, Mifepristone, Mirabegron, Mirtazapine, Moexipril/HCTZ, Moxifloxacin, Nelfinavir, Nicardipine, Nilotinib, Norepinephrine, Norfloxacin, Nortriptyline, Ofloxacin, Olanzapine, Ondansetron, Oxytocin, Paliperidone, Paroxetine, Pasireotide, Pazopanib, Pentamidine, Perflutren lipid microspheres, Phentermine, Phenylephrine, Phenylpropanolamine, Pimozide, Posaconazole, Probucol, Procainamide, Promethazine, Protriptyline, Pseudoephedrine, Quetiapine, Quinidine, Quinine sulfate, Ranolazine, Rilpivirine, Risperidone, Ritodrine, Ritonavir, Roxithromycin, Salbutamol, Salmeterol, Saquinavir, Sertindole, Sertraline, Sevoflurane, Sibutramine, Solifenacin, Sorafenib, Sotalol, Sparfloxacin, Sulpiride, Sunitinib, Tacrolimus, Tamoxifen, Telaprevir, Telavancin, Telithromycin, Terbutaline, Terfenadine, Tetrabenazine, Thioridazine, Tizanidine, Tolterodine, Toremifene, Trazodone, Trimethoprim-Sulfa, Trimipramine, Vandetanib, Vardenafil, Vemurafenib, Venlafaxine, Voriconazole, Vorinostat, or Ziprasidone.

In another embodiment, the present invention includes a method for inhibiting or decreasing the impairment of systolic ejection fraction associated with cardiotoxic chemotherapeutic treatment in a subject receiving a cardiotoxic chemotherapeutic agent causing impaired systolic ejection fraction comprising: identifying a subject in need of cardioprotection from the cardiotoxic chemotherapeutic treatment; and delivering an effective amount of a phosphatidylglycerol that is cardioprotective to the heart of the subject thereby inhibiting or decreasing impaired systolic ejection fraction associated with administration of the cardiotoxic chemotherapeutic treatment to the subject. In one aspect, the phosphatidylglycerol is delivered in combination with an existing patient care paradigm for cardiovascular disease. In another aspect, the existing patient care paradigm is selected from treatment with at least one of: antracyclins, doxorubicin, dasatinib, imatinib mesylate, lapatinib, nilotinib, sorafenib, sunitinib, or trastuzumab. In another aspect, the one or more phospholipids prevent carditoxicity after the end of the cardiotoxic therapeutic treatment. In another aspect, the one or more phospholipids is provided at least one of: before, during, or after the cardiotoxic therapeutic treatment. In another aspect, the phosphatidylglycerol is delivered at the same time as administration of the cardiotoxic chemotherapeutic agent. In another aspect, the phosphatidylglycerol inhibits at least one of pericardial fibrosis, endomyocardial fibrosis, heart failure, hemorrhagic myocardial necrosis, cardiomyopathy, myocarditis, reduction in left ventricular ejection fraction (LVEF), congestive heart failure (CHF), acute coronary disease, hypertension, myocardial infarction, or pericarditis. In another aspect, the cardiotoxic chemotherapeutic treatment is chemotherapy with sunitinib and doxorubicin. In another aspect, the phosphatidylglycerol containing compounds comprises 1,2-Dimyristoyl-sn-glycero-3-phosphorylglycerol (DMPG). In another aspect, the cardiotoxic therapeutic is a tyrosine kinase inhibitor. In another aspect, the tyrosine kinase inhibitor is selected from the group consisting of canertinib (CI 1033), erlotinib, gefitinib, imatinib mesylate, leflunomide (SU101), lapatinib, semaxinib (SU5416), sorafenib (BAY 43-9006), sunitinib, vatalanib (PTK787/ZK222584), vandetanib; ZD6474), and combinations thereof. In another aspect, the cardiotoxic chemotherapeutic treatment is a radiotherapeutic agent selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof. In another aspect, the cardiotoxic chemotherapeutic is monoclonal antibody is selected from the group consisting of, alemtuzumab, bevacizumab, cetuximab, gemtuzumab, panitumumab, rituximab, tositumomab, trastuzumab and combinations thereof In another aspect, the cardiotoxicity reduced or mitigated is at least one of: a decrease in the left ventricular ejection fraction, ejection velocity, chronic heart failure, or congestive heart failure. In another aspect, the phosphatidylglycerol does not encapsulate the cardiotoxic chemotherapeutic agent.

In another embodiment, the present invention includes a composition comprising: a therapeutically effective amount of an agent to treat a disease or condition, wherein the agent is also cardiotoxic; and a therapeutically effective amount of a phospholipid that inhibits or decreases an impaired systolic ejection fraction associated with administration of the cardiotoxic therapeutic treatment to a subject. In another aspect, the cardiotoxic therapeutic treatment is chemotherapy. In another aspect, the phospholipid is a phosphatidylglycerol is delivered in combination with an existing patient care paradigm for cardiovascular disease. In another aspect, the one or more phospholipids prevent carditoxicity after the end of the cardiotoxic therapeutic treatment. In another aspect, the one or more phospholipids is provided at least one of: before, during, or after the cardiotoxic therapeutic treatment. In another aspect, the agent is at least one of: antracyclins, doxorubicin, dasatinib, imatinib mesylate, lapatinib, nilotinib, sorafenib, sunitinib, or trastuzumab. In another aspect, the cardiotoxic therapeutic treatment is a radiotherapeutic agent is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof. In another aspect, the phospholipid is a phosphatidylglycerol is delivered at the same time as administration of the cardiotoxic therapeutic treatment. In another aspect, the phospholipid is a phosphatidylglycerol inhibits at least one of pericardial fibrosis, endomyocardial fibrosis, heart failure, hemorrhagic myocardial necrosis, cardiomyopathy, myocarditis, reduction in left ventricular ejection fraction (LVEF), congestive heart failure (CHF), acute coronary disease, hypertension, myocardial infarction, or pericarditis. In another aspect, the cardiotoxic therapeutic treatment is chemotherapy with sunitinib and doxorubicin. In another aspect, the phospholipid is a phosphatidylglycerol containing compounds comprises 1,2-Dimyristoyl-sn-glycero-3-phosphorylglycerol (DMPG). In another aspect, the cardiotoxic therapeutic treatment is with a tyrosine kinase inhibitor. In another aspect, the cardiotoxic therapeutic treatment is a tyrosine kinase inhibitor is selected from the group consisting of canertinib (CI 1033), erlotinib, gefitinib, imatinib mesylate, leflunomide (SU101), lapatinib, semaxinib (SU5416), sorafenib (BAY 43-9006), sunitinib, vatalanib (PTK787/ZK222584), vandetanib; ZD6474), and combinations thereof. In another aspect, the therapeutic treatment is a radiotherapeutic agent is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof. In another aspect, the cardiotoxic therapeutic treatment is monoclonal antibody is selected from the group consisting of, alemtuzumab, bevacizumab, cetuximab, gemtuzumab, panitumumab, rituximab, tositumomab, trastuzumab and combinations thereof. In another aspect, the cardiotoxicity reduced or mitigated is at least one of: a decrease in the left ventricular ejection fraction, ejection velocity, chronic heart failure, or congestive heart failure. In another aspect, the phospholipid is a phosphatidylglycerol does not encapsulate the cardiotoxic chemotherapeutic agent.

In another embodiment, the present invention includes a method for preventing or decreasing post-chemotherapeutic cardiotoxicity in a subject comprising: identifying a subject in need of cardioprotection from the cardiotoxic effects of a chemotherapeutic agent or treatment; and delivering an effective amount of one or more phospholipids that is cardioprotective to the heart of the subject thereby inhibiting or decreasing impaired systolic ejection fraction associated with chemotherapeutic cardiotoxicity.

In another embodiment, the present invention includes a method of evaluating a candidate drug believed to be useful in treating cardiotoxicity caused by a therapeutic agent, the method comprising: (a) measuring the cardiotoxicity from a set of patients; (b) administering a candidate drug to a first subset of the patients, and a placebo to a second subset of the patients; (c) repeating step (a) after the administration of the candidate drug or the placebo; and (d) determining if the candidate drug reduces the cardiotoxicity caused by the therapeutic agent that is statistically significant as compared to any reduction occurring in the second subset of patients, wherein a statistically significant reduction indicates that the candidate drug is useful in treating said disease state.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows that the present invention prevents the QT prolongation resulting from sunitinib administration. In FIG. 1, QT intervals were measured at specified intervals using skin surface electrodes. QT intervals were corrected as per Bazett's formula, and *indicates statistically significant differences between Sunitinib-only (left) animals and Sunitinib+1,2-dimyristoyl-sn-glycero-3-phosphorylglycerol (DMPG) (right) animals.

FIG. 2 is a graph that shows that the present invention prevents the increase in mean arterial pressure resulting from sunitinib administration.

FIG. 3 is a graph that shows that the present invention limits left ventricular hypertrophy in sunitinib-treated animals.

FIGS. 4A and 4B are graphs that show that co-treatment with sunitinib and the present invention limits the left ventricular distension associated with early-phase heart failure.

FIGS. 5A and 5B are graphs that show that the present invention limits the decrease in LV ejection velocity associated with sunitinib treatment.

FIG. 6 is a graph that shows that the present invention prevents the decrease in end-systolic left ventricular pressure-induced by sunitinib.

FIG. 7 is a graph that shows that treatment with the present invention prevents the loss in left-ventricular fractional shortening associated with sunitinib administration.

FIG. 8 is a graph that shows that treatment with the present invention prevents the weight loss observed in animals over the duration of the treatment with sunitinib.

FIG. 9 is a graph that shows the results from the co-administration of sunitinib and the invention results in significantly lower levels.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention comprises providing a lipid that inhibits drug induced cardiotoxicity, including hypertrophy, distension, atrioventricular block (AV Block), and other cardiopathies, which lipid can be provided prior to the cardiotoxic drug by, e.g., oral, parenteral (intravenous or subcutaneous) administration, or the lipid may be provided as an empty liposome prior to, concomitantly, or sequentially with therapeutic agents known to exhibit a risk of cardiotoxicity.

As used herein, the term “lipid” refers to lipids, for example, phospholipids, with the optional addition therewith of a sterol, especially cholesterol. The lipids can be provided alone or in combination with other lipids, can be saturated and unsaturated, branched or unbranched, can be in the form of a lipid tri-glycerol molecule. Non-limiting examples of phospholipids for use with the present invention include but are not limited to, e.g., 1,2-Dimyristoyl-sn-glycero-3-phosphorylcholine (DMPC), 1,2-Dimyristoyl-sn-glycero-3-phosphorylglycerol (DMPG), DMPC/DMPG, 1-myristoyl-2-hydroxy-sn-glycero-3-phospho-(1′-rac-glycerol) (LysoPG), 1-myristoyl-2-hydroxy-sn-glycero-3-phospho-(1′-rac-glycerol) (LysoPG), 1-myristoyl-2-hydroxy-sn-glycero-3-phosphocholine (LysoPC), lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine. Other non-limiting exemplary lipids for use with the present invention include, e.g., phosphatidylcholine, a phosphatidylethanolamine, a phosphatidylserine, a phosphatidylglycrol, a cardiolipin, a phosphatidylinositol or a precursor thereof in lipid, liposome, or lyso form. Non-limiting examples of lipids include lysophosphatidylglycerols for use with the present invention include lysophosphatidylcholines, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine. Asymmetric phosphatidylcholines are referred to as 1-acyl, 2-acyl-sn-glycero-3-phosphocholines, wherein the acyl groups are different from each other. Symmetric phosphatidylcholines are referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine is abbreviated herein as “DMPC.” The phosphatidylcholine 1,2-dioleoyl-sn-glycero-3-phosphocholine is abbreviated herein as “DOPC.” The phosphatidylcholine 1,2-dipalmitoyl-sn-glycero-3-phosphocholine is abbreviated herein as “DPPC.” The single fatty acid chain version of these short or long chain fatty acids are referred to as the “lyso” forms when only a single fatty acid chain is attached to the glyceryl backbone. Following the guidance of the present invention, other lipids can be identified that have the claimed function as taught herein without undue experimentation.

In one embodiment, the lysophosphatidylglycerol has a basic structure:

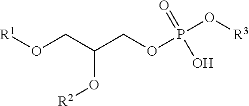

wherein R¹ or R² can be any even or odd-chain fatty acid, and R³ can be H, acyl, alkyl, aryl, amino acid, alkenes, alkynes, and wherein a short chain fatty acid is up to 5 carbons, a medium chain is 6 to 12 carbons, a long chain is 13-21 carbons and a very long chain fatty acid is greater than 22 carbons, including both even and odd chain fatty acids. In one example, the fatty acids have 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 or long fatty acids, which can be saturated or unsaturated.

In another embodiment, the phosphatidylglycerol has the basic structure:

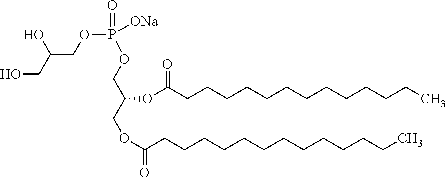

wherein such compound may optionally include an acetyl moiety attached to one or more of the hydroxyl groups.

The term “liposome” refers to a capsule wherein the wall or membrane thereof is formed of lipids, especially phospholipid, with the optional addition therewith of a sterol, especially cholesterol. In one specific non-limiting example the liposomes are empty liposomes and can be formulated from a single type of phospholipid or combinations of phospholipids. The empty liposomes can further include one or more surface modifications, such as proteins, carbohydrates, glycolipids or glycoproteins, and even nucleic acids such as aptamers, thio-modified nucleic acids, protein nucleic acid mimics, protein mimics, stealthing agents, etc. Non-limiting examples of empty liposomes for use with the present invention include but are not limited to, e.g., 1,2-Dimyristoyl-sn-glycero-3-phosphorylcholine (DMPC), 1,2-Dimyristoyl-sn-glycero-3-phosphorylglycerol (DMPG), DMPC/DMPG, 1-myristoyl-2-hydroxy-sn-glycero-3-phospho-(1′-rac-glycerol) (LysoPG), and 1-myristoyl-2-hydroxy-sn-glycero-3-phospho-(1′-rac-glycerol) (LysoPG). In one embodiment, the liposome is a liposome or a liposome precursor comprising, e.g., a LysoPG, a myristoyl monoglyceride, and a myristic acid. In one specific, non-limiting example the composition also comprises an active agent in or about the liposome and the composition has a ratio of phospholipids to active agent of 3:1, 1:1, 0.3:1, and 0.1:1.

In one embodiment, the lipid has the following structural formula:

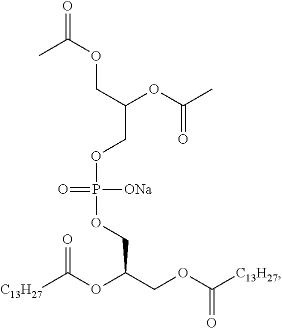

salts or solvates thereof;

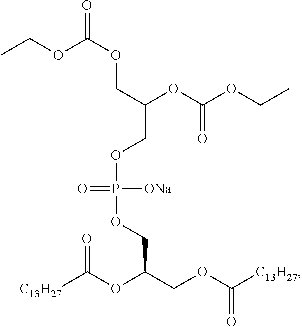

salts or solvates thereof;

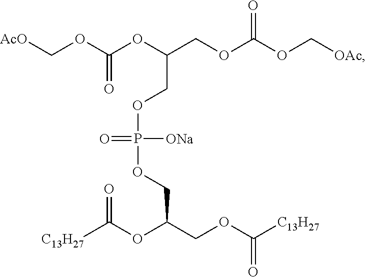

salts or solvates thereof;

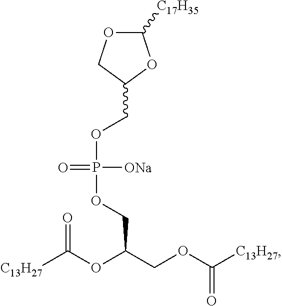

salts or solvates thereof;

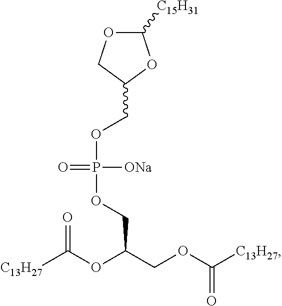

salts or solvates thereof; or

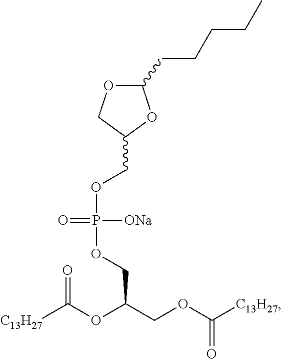

salts or solvates thereof.

As used herein, the term “in vivo” refers to being inside the body. The term “in vitro” used as used in the present application is to be understood as indicating an operation carried out in a non-living system.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology); or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the terms “effective amount” or “therapeutically effective amount” described herein means the amount of the subject compound that will elicit the biological or medical response of a tissue, system, animal or human that is being sought by the researcher, veterinarian, medical doctor or other clinician.

As used herein, the terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

As used herein the term “intravenous administration” includes injection and other modes of intravenous administration.

As used herein, the term “pharmaceutically acceptable” as used herein to describe a carrier, diluent or excipient must be compatible with the other ingredients of the formulation and not deleterious to the recipient thereof.

Channelopathies. The human ether-à-go-go gene related cardiac tetrameric potassium channel, when mutated, can render patients sensitive to over 163 drugs which may inhibit ion conduction and deregulate action potentials. Prolongation of the action potential follows effects in the potassium channel. Ion channel active drugs may directly increase the QTc interval, and increase the risk of torsade de pointes and sudden cardiac death. Exacerbation of cardiomyocyte potassium channel sensitivity to drugs may also be associated with metabolic diseased states including diabetes or may be of idiopathic origin.

For these reasons, evaluation of drug effects on cardiac potassium channel function is a critical step during drug development, and when serious, may be an obstacle to regulatory approval. In whole-cell patch-clamp experiments, curcumin inhibited hERG K⁺ currents in HEK293 cells stably expressing hERG channels in a dose-dependent manner with IC₅₀ value of 5.55 μM. The deactivation, inactivation and the recovery time from inactivation of hERG channels were significantly changed by acute treatment with 10 μM curcumin. Incubation of 20 μM curcumin for 24 h reduced the HEK293 cell viability. Intravenous injection of 20 mg of curcumin in rabbits did not affect the cardiac repolarization reflected by QTc values. (Hu CW 2012). These molecules are specific liposomes, or components of liposomes that were initially bound to lipophilic drugs to permit intravenous solubility at physiological conditions, and reduce adverse events. The loci of action appears to be in intra-channel ion selectivity or gating site(s) controlling potassium ion movement: a key functional component of regulation of action potentials which lead downstream to myocyte contraction.

By way of explanation, and in no way a limitation of the present invention, the mechanism of human ether-à-go-go related gene channels blockade may be analogous to the effects of externally applied quaternary ammonium derivatives which indirectly may suggest the mechanism of action of the anti-blockading effect of the DMPC/DMPG liposome or its metabolites. The inhibitory constants and the relative binding energies for channel inhibition indicate that more hydrophobic quaternary ammoniums have higher affinity blockade while cation-it interactions or size effects are not a deterministic factor in channel inhibition by quaternary ammoniums. Also hydrophobic quaternary ammoniums either with a longer tail group or with a bigger head group than tetraethylammonium permeate the cell membrane to easily access the high-affinity internal binding site in the gene channel and exert a stronger blockade.

These data show that the basis for the ameliorating effect liposome, or its components is the higher competitive affinity for binding sites by the DMPC and DMPG compared to QTc prolonging drugs, its constitutive lack of ion transport modulation, i.e. liposome or its fragments do not impede K+ ion transport.

Again, by way of explanation and in no way a limitation of these claims, these data show that the basis for the ameliorating effect liposome, or its components, is the higher competitive affinity for binding sites by the DMPC and DMPG compared to QTc prolonging drugs, its constitutive lack of ion transport modulation, i.e., liposome, or its fragments, do not impede K+ ion transport, and indicates that the site of the mechanism of DMPC or DMPG protection may be in the selectivity segment of the channel or in the hydration surrounding the ion. Additionally, based upon these hERG channel data, the structures of these liposome components may be informative for designing or selecting other molecules to prevent drug induced cardiac arrhythmias.

Naive adult male Hartley guinea pigs weighing between 0.40 kg and 0.50 kg were treated with either sunitinib (10 mg/kg/day) for 28 days, followed by 15 days of rest, another 28-day cycle, followed by a last 15-day washout period. The treatments were accompanied, or not, by the invention, at a dose of 10 mg/kg/day mg/kg/day. This was designed to mimic a common cycle of chemotherapy in humans. Body weights were measured weekly, as well as food consumption. Blood draws, and echocardiographies were obtained on Day 0 (prior to treatment), on Day 43 (end of resting period, between-cycles) and on Day 86. Systemic arterial blood pressure was measured invasively on Day 86 only. Troponins I and T, as well as CKMB (phosphocreatine kinase-cardiac isoform) were quantified from the blood, while echocardiography data was analyzed for right and left ventricular volumes, and ejection kinetics. The heart of each animal was mounted on a Langendorff retrograde perfusion system to measure left ventricle contractility and kinetics.

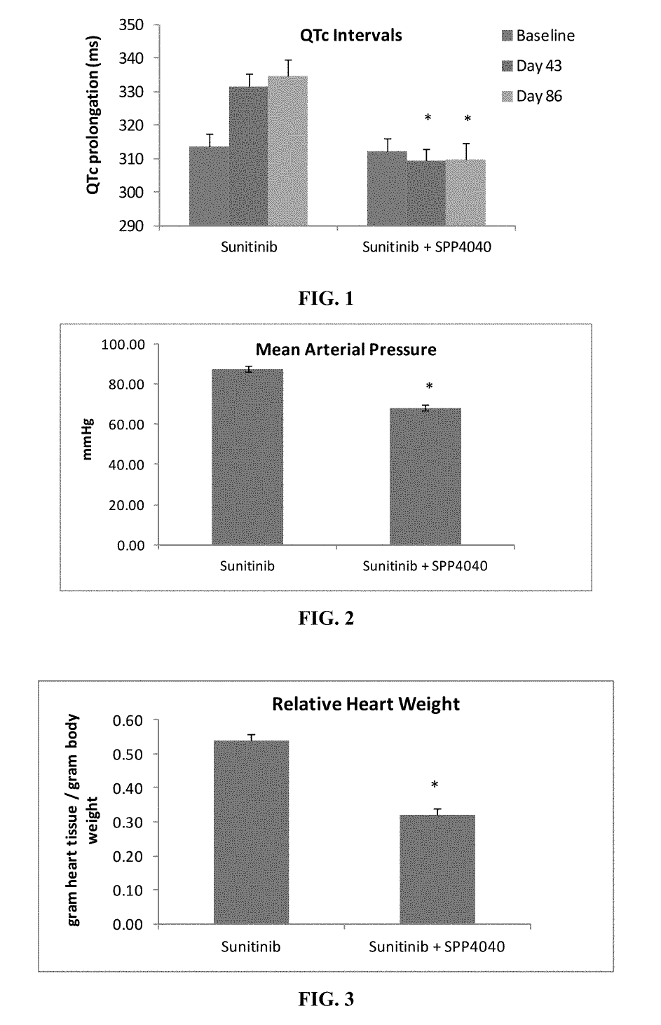

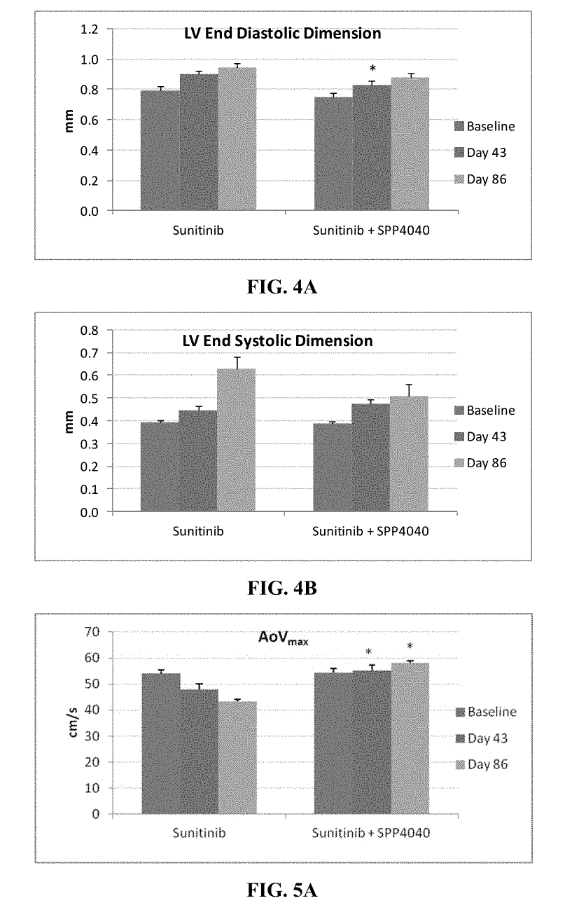

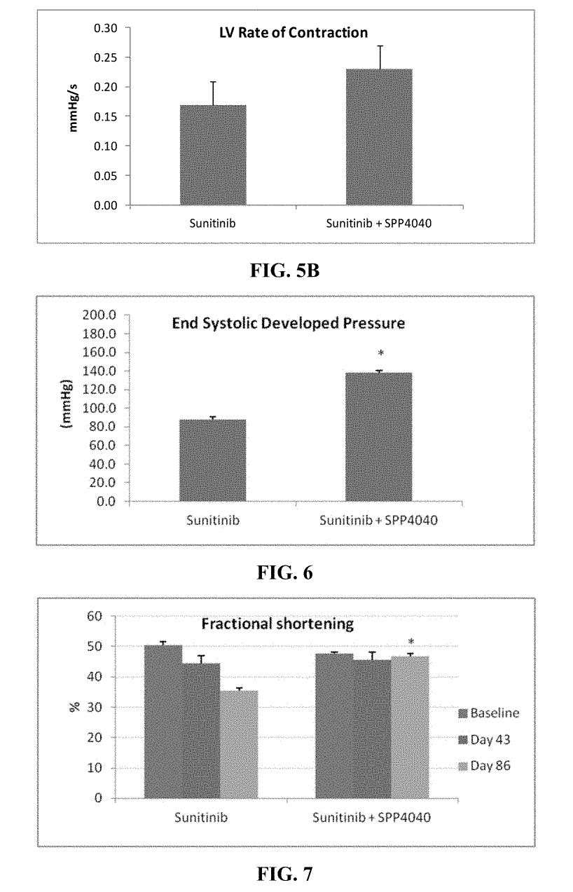

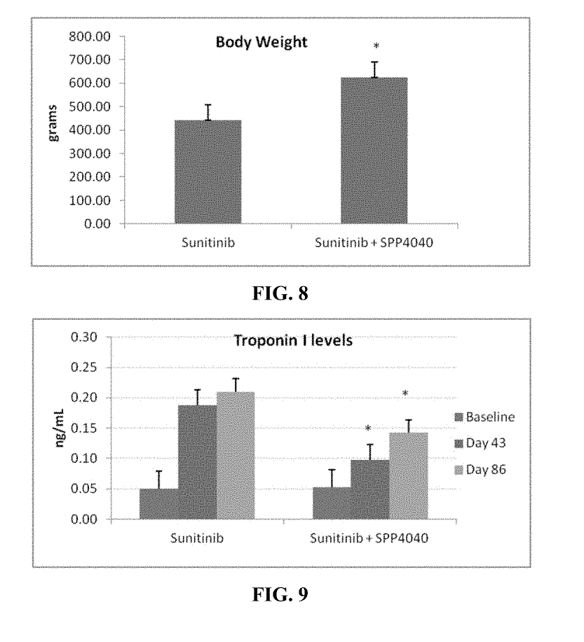

Results. The animals (n=6 per group) exposed to sunitinib, compared to sunitinib co-administered with the invention, exhibited the following symptoms:
1. 15-25 ms QTc prolongation as of Day 28, increasing steadily until Day 86 (sacrifice) (FIG. 1)
2. Significantly higher mean arterial blood pressure (FIG. 2)
3. The chronic hypertension led to congestive heart failure, characterized by:
   a. left-ventricular distention with onset of hypertrophy (FIGS. 3, 4)
   b. lower ejection velocity (FIG. 5)
   c. lower end-systolic left ventricular pressure (FIG. 6)
   d. lower left-ventricular fractional shortening (FIG. 7)
4. Significant weight loss over the duration of the treatment (FIG. 8)
5. Significantly higher troponin I levels, indicative of myocardial damage (FIG. 9).

In comparison, the animals co-administered with sunitinib and the invention exhibited
1. No change in QTc intervals vs. Day 0 data.
2. Lower mean arterial pressure, less likely to lead to congestive heart dysfunction
3. Significantly lower symptoms of congestive heart failure, including hypertrophy, changes in left-ventricular kinetics, and weight loss.

FIG. 1 is a graph that shows that the present invention prevents the QT prolongation resulting from sunitinib administration. In FIG. 1, QT intervals were measured at specified intervals using skin surface electrodes. QT intervals were corrected as per Bazett's formula.* indicate statistically significant differences between Sunitinib-only (left) animals and Sunitinib+1,2-dimyristoyl-sn-glycero-3-phosphorylglycerol (DMPG) (right) animals.

FIG. 2 is a graph that shows that the present invention prevents the increase in mean arterial pressure resulting from sunitinib administration. In FIG. 2, mean arterial pressure was measured invasively by inserting a catheter-mounted pressure tranducer into the femoral artery of the anesthetized animals, on Day 86. Sunitinib+1,2-dimyristoyl-sn-glycero-3-phosphorylglycerol (DMPG) animals exhibited a significantly lower mean arterial pressure than those animals receiving sunitinib alone.

FIG. 3 is a graph that shows that the present invention limits left ventricular hypertrophy in sunitinib-treated animals. In FIG. 3, Sunitinib caused cardiac hypertrophy after 86 days (2 cycles) of treatment. An increase in left-ventricular size (distension) drove the increase in overall heart weight. In contrast, the animals treated with Sunitinib and 1,2-dimyristoyl-sn-glycero-3-phosphorylglycerol (DMPG) exhibited significantly less gain in cardiac weight.

FIGS. 4A and 4B are graphs that show that co-treatment with sunitinib and the present invention limits the left ventricular distension associated with early-phase heart failure. In FIGS. 4A and 4B, early stages of heart failure are characterized by LV distension. Sunitinib alone caused a greater LV distension than Sunitinib+1,2-dimyristoyl-sn-glycero-3-phosphorylglycerol (DMPG), as measured at the end of the diastole (A: i.e. after complete filling of the left ventricle) and at the end of the systole (B: once the ventricle has emptied out its content into the aorta). As the treatment progresses, the failing LV becomes more distended: the invention limits the distension of the LV caused by suninitib.

FIGS. 5A and 5B are graphs that show that the present invention limits the decrease in LV ejection velocity associated with sunitinib treatment. In FIG. 5A, a gradual decrease in LV ejection velocity at the aortic valve *(AoVMax) could be measured by cardiac echography in sunitinib-treated animals. The decrease in ejection velocity is characteristic of early-stage LV failure. Those animals receiving a combination of the invention and sunitinib did not exhibit any decrease in ejection velocity. In FIG. 5B, on Day 86, the animals were euthanized and the hearts mounted onto a Langendorff retrograde perfusion system. A left ventricular pressure transducer was inserted into the left ventricle and recorded LV contraction amplitude and kinetics. The rate of contraction of the LV was lower in sunitinib-only animals, compared to animals treated with a combination of the invention and sunitinib.

FIG. 6 is a graph that shows that the present invention prevents the decrease in end-systolic left ventricular pressure-induced by sunitinib. In FIG. 6, the pressure developed by the heart on Day 86 of treatment was measured ex-vivo in a Langendorff retrograde perfusion system. Those hearts from animals treated with sunitinib only exhibited a significantly lower developed LV pressure (lower contractile force) than those hearts from animals treated with the combination of sunitinib and the invention.

FIG. 7 is a graph that shows that treatment with the present invention prevents the loss in left-ventricular fractional shortening associated with sunitinib administration. In FIG. 7, a loss in LV fractional shortening results from early-stage myocardial remodeling. Animals treated with sunitinib only exhibited a time-dependent loss in LV fractional shortening leading to decreasing LV ejection fraction, which was not observed in the animals treated with sunitinib and the invention. The difference between the two groups of animals was statistically significant after 86 days of treatment.

FIG. 8 is a graph that shows that treatment with the present invention prevents the weight loss observed in animals over the duration of the treatment with sunitinib. In FIG. 8, a common indicator of well-being, or inversely, of discomfort in laboratory animals is weight loss. The animals treated with sunitinib exhibited limited gains in body weight over the 86 days of treatment, while the animals co-treated with sunitinib and the invention exhibited a statistically greater gain in body weight, suggesting a lower level of discomfort associated with the treatment.

FIG. 9 is a graph that shows the results from the co-administration of sunitinib and the invention results in significantly lower levels. FIG. 9 shows that in heart failure, stretch due to myocardial overload can induce myocyte necrosis and apoptosis, releasing troponins T and I. Troponin I is generally considered more sensitive, and was used as a biomarker of acute and chronic myocardial distress. Those animals treated with sunitinib alone produced levels of Troponin I, which were significantly higher than those animals that were treated with the invention and sunitinib. Furthermore, the animals treated with sunitinib alone presented troponin levels which were significantly greater than the levels measured in the intact animals (0.05 ng/mL, data not shown).

Similar results were obtained with naive, adult, male Sprague-Dawley rats. The guinea pig was used as a test species in this development program because it exhibits ECG signals, which are cleaner than those of rats, especially the T-wave which is necessary for precise QT-interval measurements.

Similar results were obtained when the animals (guinea pigs) were exposed to 1.5 dmg/kg/day doxorubicin, alone or co-administered with the invention, for the same treatment duration.

These results indicate that co-administration of sunitinib and doxorubicin with the cardioprotective phosphatidylglycerols effectively mitigates, and even suppresses, the cardiac adverse effects associated with cardiotoxic chemotherapy agents.

Thus, in one non-limiting example, the co-administration of these chemotherapy agents with the invention leads to faster patient recovery as a result of more aggressive therapeutic dosage, because such dosage is currently limited by the cardiac adverse effects experienced by the patients.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 8,372,830.

https://www.ncbi.nlm.nih.gov/pmc/articles/PMC4168851/

https://academic.oup.com/toxsci/article/120/1/14/1665205/Cardiotoxicity-Associated-with-Targeting-Kinase

The importance of drug metabolites synthesis: the case-study of cardiotoxic anticancer drugs. Hrynchak I, Sousa E, Pinto M, Costa V M. Drug Metab Rev. 2017; 25:1-39

Cardiac Complications of Cancer Therapy: Pathophysiology, Identification, Prevention, Treatment, and Future Directions. Jain D, Russell R R, Schwartz R G, Panjrath G S, Aronow W. Curr Cardiol Rep. 2017; 19(5):36.

Beyond Anthracyclines: Preemptive Management of Cardiovascular Toxicity in the Era of Targeted Agents for Hematologic Malignancies. Sethi T K, Basdag B, Bhatia N, Moslehi J, Reddy N M. Curr Hematol Malig Rep. 2017; 12(3):257-267.

The Myocyte-Damaging Effects of the BCR-ABL1-Targeted Tyrosine Kinase Inhibitors Increase with Potency and Decrease with Specificity. Hasinoff B B, Patel D, Wu X. Cardiovasc Toxicol. 2016.

Validating the pharmacogenomics of chemotherapy-induced cardiotoxicity: What is missing? Magdy T, Burmeister B T, Burridge P W. Pharmacol Ther. 2016; 168:113-125.

Progress on the cardiotoxicity of sunitinib: Prognostic significance, mechanism and protective therapies. Yang Y, Bu P. Chem Biol Interact. 2016; 25;257:125-31.

Cardiovascular Complications of Targeted Therapies for Chronic Myeloid Leukemia. Damrongwatanasuk R, Fradley M G. Curr Treat Options Cardiovasc Med. 2017; 19(4):24.

Left ventricular dysfunction predicted by early troponin I release after high-dose chemotherapy. Cardinale D, Sandri M T, Martinoni A, Tricca A, Civelli M, Lamantia G, Cinieri S, Martinelli G, Cipolla C M, Fiorentini C. J Am Coll Cardiol. 2000; 36(2):517-22.

Orphanos G S, I. G. (2009). Cardiotoxicity induced by tyrosine kinase inhibitors. Acta Oncol., 48 (7), pp. 964-970.

What is claimed is:

1. A method for inhibiting or decreasing impaired systolic ejection fraction associated with cardiotoxic therapeutic treatment in a subject receiving a cardiotoxic chemotherapeutic agent causing impaired ejection fraction comprising:
identifying a subject in need of cardioprotection from the cardiotoxic therapeutic agent or treatment; and
delivering an effective amount of one or more phospholipids that is cardioprotective to the heart of the subject thereby inhibiting or decreasing impaired systolic ejection fraction associated with administration of the cardiotoxic therapeutic treatment to the subject.

2. The method of claim 1, wherein the cardiotoxic therapeutic treatment is chemotherapy, wherein the one or more phospholipids is provided at least one of: before, during, or after the cardiotoxic therapeutic treatment, or wherein the one or more phospholipids is a phosphatidylglycerol that is delivered in combination with an active agent that also treats the cardiovascular disease.

3. The method of claim 2, wherein the cardiotoxic therapeutic treatment is selected from treatment with at least one of: antracyclins, doxorubicin, dasatinib, imatinib mesylate, lapatinib, nilotinib, sorafenib, sunitinib, sunitinib and doxorubicin, or trastuzumab, or is a tyrosine kinase inhibitor is selected from the group consisting of canertinib (CI 1033), erlotinib, gefitinib, imatinib mesylate, leflunomide (SU101), lapatinib, semaxinib (SU5416), sorafenib (BAY 43-9006), sunitinib, vatalanib (PTK787/ZK222584), vandetanib; ZD6474), and combinations thereof, or is a monoclonal antibody selected from the group consisting of, alemtuzumab, bevacizumab, cetuximab, gemtuzumab, panitumumab, rituximab, tositumomab, trastuzumab and combinations thereof.

4. The method of claim 1, wherein the one or more phospholipids is a phosphatidylglycerol that inhibits at least one of pericardial fibrosis, endomyocardial fibrosis, heart failure, hemorrhagic myocardial necrosis, cardiomyopathy, myocarditis, reduction in left ventricular ejection fraction (LVEF), congestive heart failure (CHF), acute coronary disease, hypertension, myocardial infarction, or pericarditis.

5. The method of claim 1, wherein the one or more phospholipids is a phosphatidylglycerol containing compounds comprises 1,2-Dimyristoyl-sn-glycero-3-phosphorylglycerol (DMPG).

6. The method of claim 1, wherein the cardiotoxic therapeutic treatment is a radiotherapeutic agent is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof.

7. The method of claim 1, wherein the cardiotoxicity reduced or mitigated is at least one of: a decrease in the left ventricular ejection fraction, ejection velocity, chronic heart failure, or congestive heart failure.

8. The method of claim 1, wherein the phospholipid does not encapsulate the cardiotoxic therapeutic agent.

9. The method of claim 1, wherein the phospholipid has the following structural formula: 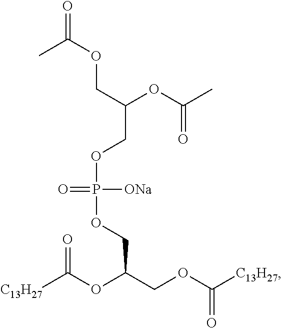
salts or solvates thereof; 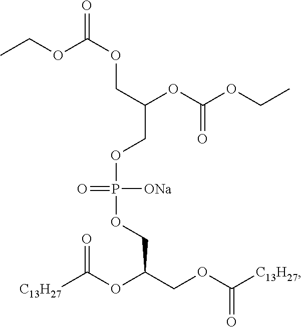
salts or solvates thereof; 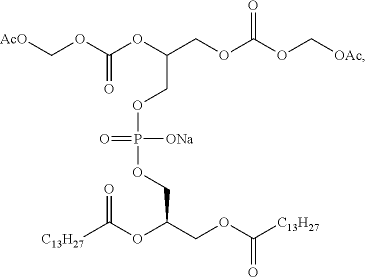
salts or solvates thereof; 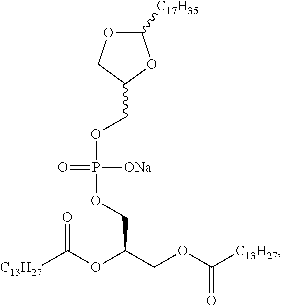
salts or solvates thereof; 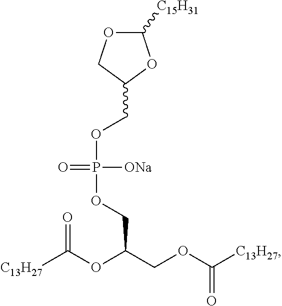
salts or solvates thereof; or 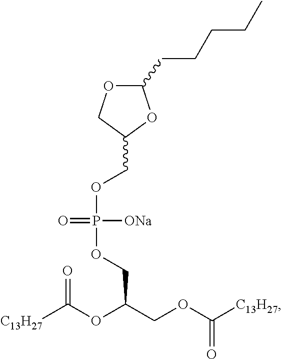
salts or solvates thereof.

10. A method for inhibiting or decreasing impaired systolic ejection fraction associated with cardiotoxic chemotherapeutic treatment in a subject receiving a cardiotoxic chemotherapeutic agent causing impaired systolic ejection fraction comprising:
identifying a subject in need of cardioprotection from the cardiotoxic chemotherapeutic treatment; and
delivering an effective amount of a phosphatidylglycerol that is cardioprotective to the heart of the subject thereby inhibiting or decreasing impaired systolic ejection fraction associated with administration of the cardiotoxic chemotherapeutic treatment to the subject.

11. The method of claim 10, wherein the cardiotoxic therapeutic treatment is chemotherapy, wherein the one or more phospholipids is provided at least one of: before, during, or after the cardiotoxic therapeutic treatment, or wherein the one or more phospholipids is a phosphatidylglycerol that is delivered in combination with an active agent that also treats the cardiovascular disease.

12. The method of claim 10, wherein the cardiotoxic therapeutic treatment is selected from treatment with at least one of: antracyclins, doxorubicin, dasatinib, imatinib mesylate, lapatinib, nilotinib, sorafenib, sunitinib, sunitinib and doxorubicin, or trastuzumab, or is a tyrosine kinase inhibitor is selected from the group consisting of canertinib (CI 1033), erlotinib, gefitinib, imatinib mesylate, leflunomide (SU101), lapatinib, semaxinib (SU5416), sorafenib (BAY 43-9006), sunitinib, vatalanib (PTK787/ZK222584), vandetanib; ZD6474), and combinations thereof, or is a monoclonal antibody selected from the group consisting of, alemtuzumab, bevacizumab, cetuximab, gemtuzumab, panitumumab, rituximab, tositumomab, trastuzumab and combinations thereof.

13. The method of claim 10, wherein the one or more phospholipids is a phosphatidylglycerol that inhibits at least one of pericardial fibrosis, endomyocardial fibrosis, heart failure, hemorrhagic myocardial necrosis, cardiomyopathy, myocarditis, reduction in left ventricular ejection fraction (LVEF), congestive heart failure (CHF), acute coronary disease, hypertension, myocardial infarction, or pericarditis.

14. The method of claim 10, wherein the one or more phospholipids is a phosphatidylglycerol containing compounds comprises 1,2-Dimyristoyl-sn-glycero-3-phosphorylglycerol (DMPG).

15. The method of claim 10, wherein the cardiotoxic therapeutic treatment is a radiotherapeutic agent is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof.

16. The method of claim 1, wherein the cardiotoxicity reduced or mitigated is at least one of: a decrease in the left ventricular ejection fraction, ejection velocity, chronic heart failure, or congestive heart failure.

17. The method of claim 10, wherein the phospholipid has the following structural formula: 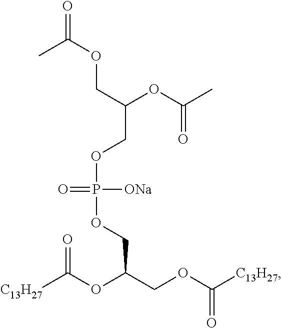
salts or solvates thereof; 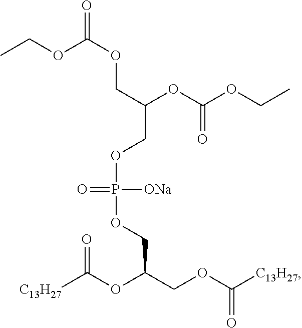
salts or solvates thereof; 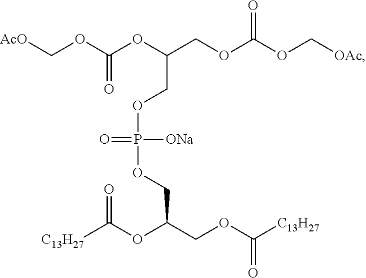
salts or solvates thereof; 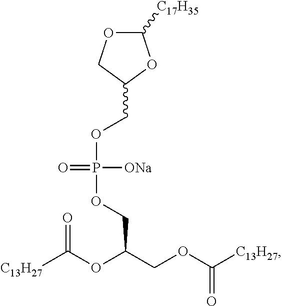
salts or solvates thereof; 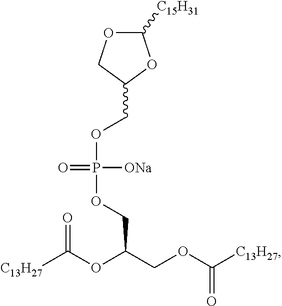
salts or solvates thereof; or 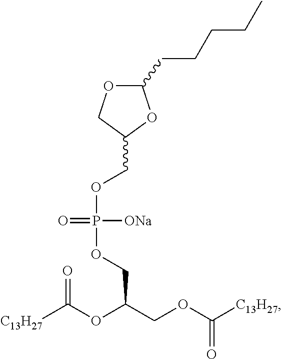
salts or solvates thereof.

18. A composition comprising:
a therapeutically effective amount of an agent to treat a disease or condition, wherein the agent is also cardiotoxic; and
a therapeutically effective amount of a phospholipid that inhibits or decreases an impaired systolic ejection fraction associated with administration of the cardiotoxic therapeutic treatment to a subject.

19. The composition of claim 18, wherein the cardiotoxic therapeutic treatment is chemotherapy, wherein the one or more phospholipids is provided at least one of: before, during, or after the cardiotoxic therapeutic treatment, or wherein the one or more phospholipids is a phosphatidylglycerol that is delivered in combination with an active agent that also treats the cardiovascular disease.

20. The composition of claim 18, wherein the cardiotoxic therapeutic treatment is selected from treatment with at least one of: antracyclins, doxorubicin, dasatinib, imatinib mesylate, lapatinib, nilotinib, sorafenib, sunitinib, sunitinib and doxorubicin, or trastuzumab, or is a tyrosine kinase inhibitor is selected from the group consisting of canertinib (CI 1033), erlotinib, gefitinib, imatinib mesylate, leflunomide (SU101), lapatinib, semaxinib (SU5416), sorafenib (BAY 43-9006), sunitinib, vatalanib (PTK787/ZK222584), vandetanib; ZD6474), and combinations thereof, or is a monoclonal antibody selected from the group consisting of, alemtuzumab, bevacizumab, cetuximab, gemtuzumab, panitumumab, rituximab, tositumomab, trastuzumab and combinations thereof.

21. The composition of claim 18, wherein the one or more phospholipids is a phosphatidylglycerol that inhibits at least one of pericardial fibrosis, endomyocardial fibrosis, heart failure, hemorrhagic myocardial necrosis, cardiomyopathy, myocarditis, reduction in left ventricular ejection fraction (LVEF), congestive heart failure (CHF), acute coronary disease, hypertension, myocardial infarction, or pericarditis.

22. The composition of claim 18, wherein the one or more phospholipids is a phosphatidylglycerol containing compounds comprises 1,2-Dimyristoyl-sn-glycero-3-phosphorylglycerol (DMPG).

23. The composition of claim 18, wherein the cardiotoxic therapeutic treatment is a radiotherapeutic agent is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and combinations thereof.

24. The composition of claim 18, wherein the cardiotoxicity reduced or mitigated is at least one of: a decrease in the left ventricular ejection fraction, ejection velocity, chronic heart failure, or congestive heart failure.

25. The composition of claim 18, wherein the phospholipid has the following structural formula: 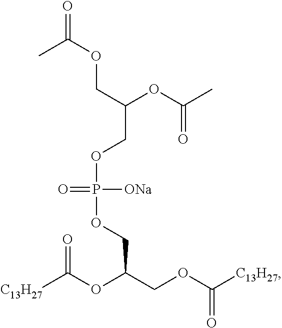
salts or solvates thereof; 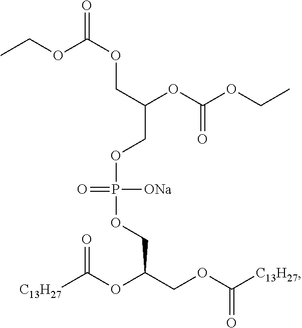
salts or solvates thereof; 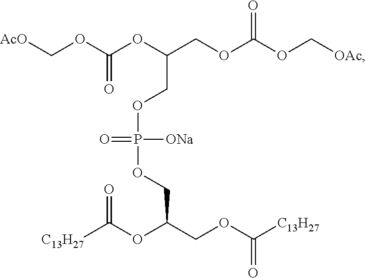
salts or solvates thereof; 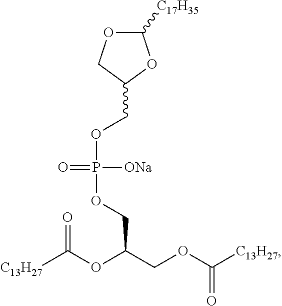
salts or solvates thereof; 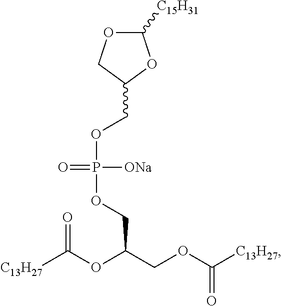
salts or solvates thereof; or 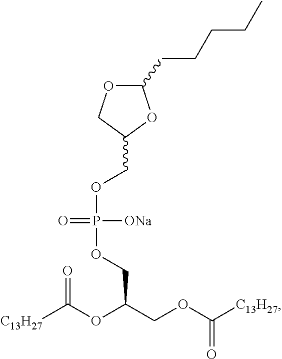
salts or solvates thereof.

26. A method of evaluating a candidate drug believed to be useful in treating cardiotoxicity caused by a therapeutic agent, the method comprising:
(a) measuring the cardiotoxicity from a set of patients;
(b) administering a candidate drug to a first subset of the patients, and a placebo to a second subset of the patients;
(c) repeating step (a) after the administration of the candidate drug or the placebo; and
(d) determining if the candidate drug reduces the cardiotoxicity caused by the therapeutic agent that is statistically significant as compared to any reduction occurring in the second subset of patients, wherein a statistically significant reduction indicates that the candidate drug is useful in treating said disease state.
